(19) United States
(12) Patent Application Publication
(10) Pub. No.: US 2004/0050391 A1
(43) Pub. Date: Mar. 18, 2004

(54) Leg assist device

(76) Inventors: Debra Kiwala, St. Louis, MO (US); Carolyn Johnson, St. Peters, MO (US)

Correspondence Address: Debra Kiwala, 4155 Rinzetta Dr, St. Louis, MO, 63129, US

(21) Appl. No.: 10/340,826

(22) Filed: Sep. 16, 2002

(51) Int. Cl. A61F 5/37

(52) U.S. Cl. 128/882

(57) ABSTRACT

The Leg Assist Device will make future medical procedures and needs safer and more user friendly. With the use of this simple device the medical field will be able to insert urinary foley catheters without the risk of patients bending their legs and contaminating the sterile field, thus cutting down risk for infection significantly. With the use of our device it will also lessen the need for staffing in this procedure since the bar holds the legs in place, thus no need for the two additional staff members to be performing this old job.

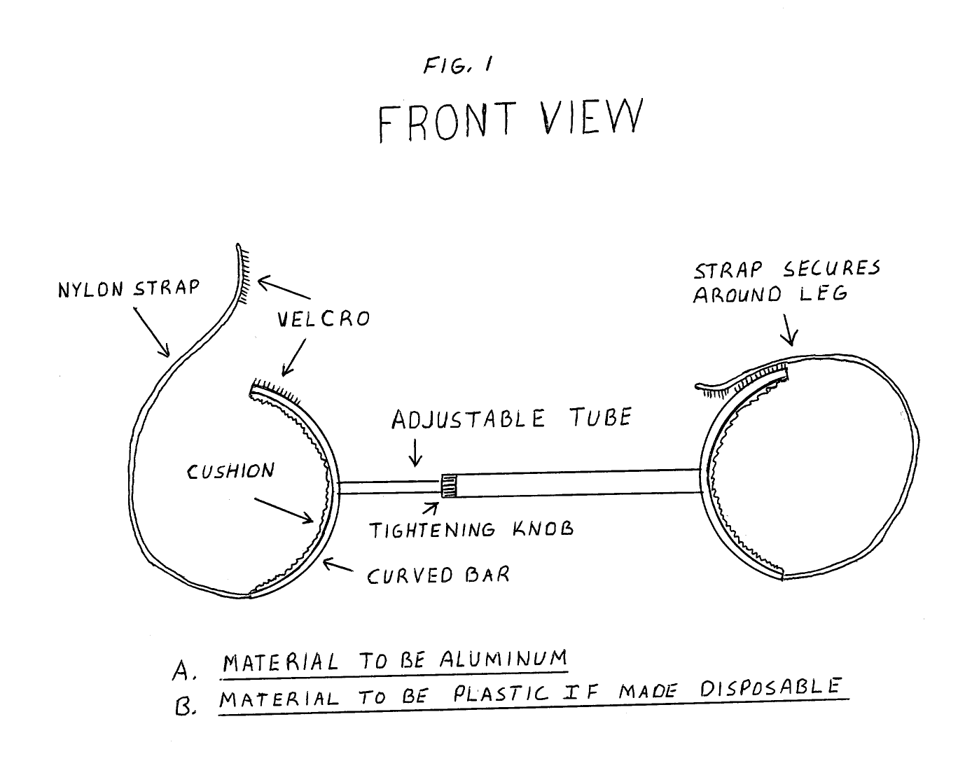

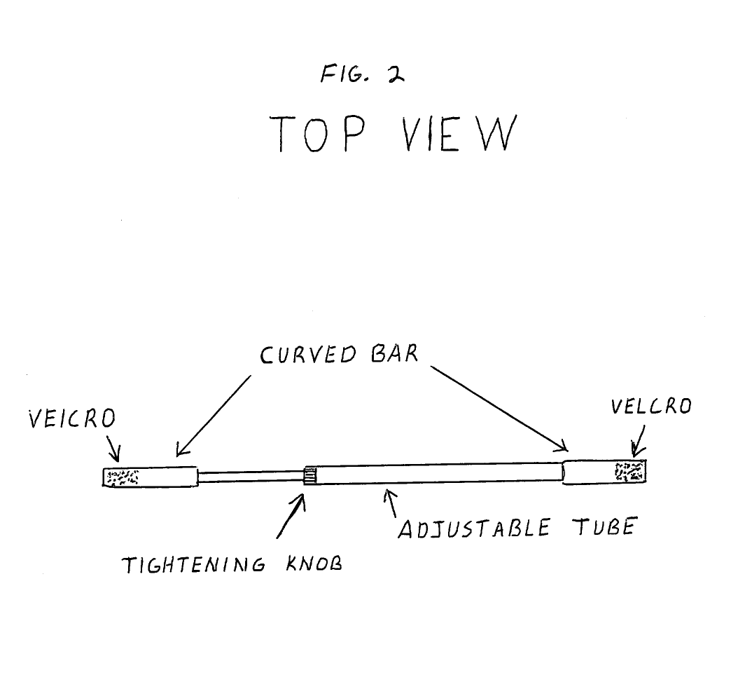

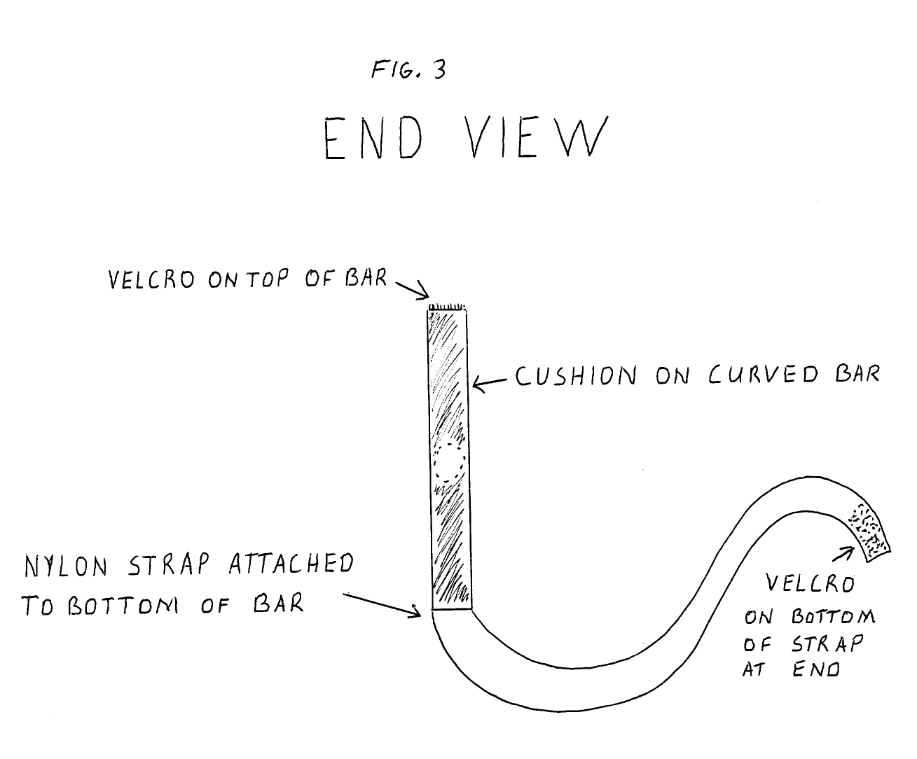

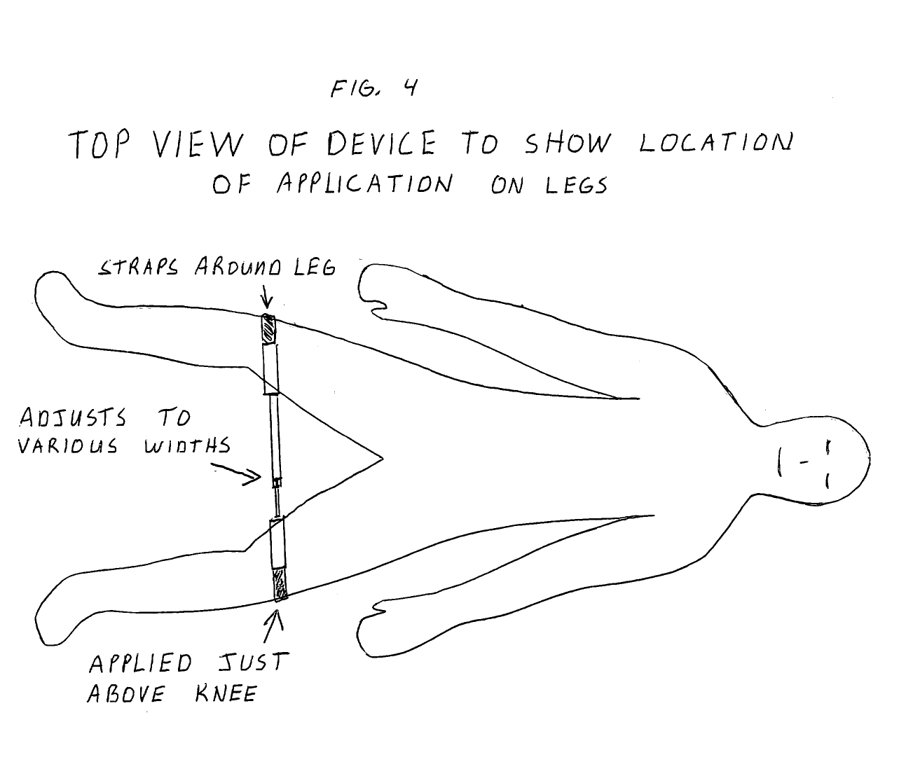

### BACKGROUND OF INVENTION

In the medical field Nurses place urinary foley catheter all the time. One day when I was at work I received an order from a Doctor to do this. We will call the pt Mrs. X. Mrs. X was a very old lady and did not have all her mental stability. She was not aware that the foley catheter was for her benefit and was not quite sure what we were doing even after much patient instruction. I recruited two other staff members to help hold Mrs. X legs apart while I tried to insert the catheter using sterile technique. One of the staff became frustrated with Mrs. X and let her leg drop onto my sterile field. I had to restart using all new materials. After much time and money the patient finally had a foley catheter and the floor was back to normal. I then thought how much easier it would have been just to have had a retractable bar in place instead of using the two other staff members. This would have saved time and money since I would not have used a second catheter set or the two other staff members. Many times Nurses do reuse their catheters after breaking sterile technique leading to many urinary infections. I thought of this device many times and how it would have made my job easier. I finally have put it down on paper and am going ahead with my idea. I know that this device will make my job and other Nurses job easier in the future. I also know this will help out my fellow Physical Therapist in the event that they have a patient after a Total Hip surgery and they need a device to help keep their patients legs apart to prevent injury while healing. At the present they are using a pillow that is shaped like a triangle. Many times this pillow has fallen out of place or it is uncomfortable to the patient since there is only one size pillow. I know this invention will help out many in the years to come.

### SUMMARY OF THE INVENTION

The Leg Assistive Device was invented for the purpose to help the Health Care worker when placing a urinary foley catheter. The device helps spread open the legs to make it easier to insert a urinary catheter. Some patients such as a paraplegic, quadriplegic, Alzheimer or other mentally challenged patients have a difficult time keeping their legs open. This results in many problem. This procedure needs to be performed with sterile technique. When sterile technique is broken the risk for infection is almost a guarantee. This device helps keep the patients legs open safely related to the spring in the middle of the bar so patients can move their legs without closing them off. The soft durable straps keep their legs in place. The other advantage to this device is that you do not need the additional staff to help assist with this procedure which would decrease employee/patient injury.

### NATURE OF INVENTION

This invention is a plastic bar ranging in size from approximately 12 to 24 inches, sizes can change related to age of patient. The bar can be adjusted using the spring like mechanism and the peg holes to help hold the desired length. The bar has plastic leg rest at each end for the persons knee to rest in. There are straps on the leg rest that can be used optionally for patients that are unable to keep their legs in place voluntarily.

Picture 1 shows a human with the device in place, in between the legs.

Picture 2 shows the device in more detail. The adjustable tube, that could be made either out of aluminum or plastic. The ends of the adjustable tube show half circles which will be holding the leg in a comfortable manner. These also can be made out of plastic or aluminum. On the top of the half circle will be a open ring to allow a Velcro strap to be pulled threw and adjusted to each patients leg size.

1. We claim our invention the “Leg assist device’ will help by spreading apart the legs of a patient this can be used in aiding for any patient needing to keep the legs apart in a safe and comfortable manner.
